(19) United States
(12) Patent Application Publication
(10) Pub. No.: US 2006/0222695 A1
(43) Pub. Date: Oct. 5, 2006

(54) Deoxycholic acid liposome-based dermatological topical preparation

(76) Inventors: Filiberto Zadini, North Hills, CA (US); Giorgio Zadini, Camarillo, CA (US)

Correspondence Address: FILIBERTO ZADINI, 2237 HILLTOP LANE, CAMARILLO, CA, 93012, US

(21) Appl. No.: 11/096,350

(22) Filed: Apr. 1, 2005

(51) Int. Cl. A61K 9/127

(52) U.S. Cl. 424/450

(57) ABSTRACT

A dermatological topical preparation such a cream, a lotion, an emulsion, a paste, an ointment and the likes including liposomes carrying lipo-dissolving substances encapsulated by the liposomes wall or incorporated with the liposomes wall components. The lipo-dissolving substance is released by the liposomes into the target adipose tissue or its proximity after penetration of the superficial skin layers by the liposomes carrying the lipo-dissolving substance.

### FIELD OF THE INVENTION

This invention relates to dermatological topical preparations for the treatment of localized unwanted adiposities.

Background—Description of the Prior Art

Numerous treatments, topical and non topical, are available today for treatment of localized adiposities and lipodystrophies. Some of these treatments have a scientific base, some a pseudo-scientific empiric base.

Among the topical treatments, only the treatments delivered via traditional injections have achieved clinically satisfactory results.

Topical treatments based on transdermal delivery of medications or cosmoceuticals using methods other than percutaneous injections have the highest rate of failure. Indeed transdermal delivery of lipo-dissolving drugs and/or cosmoceutical with topical conventional preparations such as creams, lotions, emulsions, pastes, ointments is destined to failure as only a very small percentage, if any, of such lipo-dissolving preparations can get thru the nearly impermeable barrier of the horny impermeable layer of the skin known as stratum corneum.

Due to the above mentioned physiological obstacle to deep penetration represented by the stratum corneum, chemical compounds that have shown lipo-dissolving activity when introduced via injections, have consistently failed to reproduce their lipo-dissolving activity incorporated in conventional creams, lotions, emulsions, pastes, ointments and the likes.

### BRIEF SUMMARY OF THE INVENTION

With the present invention applicants propose a topical dermatological preparation having the scientifically proven characteristics of being capable of:

A) delivering the drug or cosmoceutical into the subcutaneous tissue directly into the fat deposits or their proximity and

B) of delivering a specific chemical compound which has shown to effectively dissolve fat deposits when introduced percutaneously via injection into the fat deposits and or their proximity.

More specifically, the present invention discloses a topical preparation such as a cream, lotion, emulsion, paste, ointment and the likes based in the liposome transdermal delivery, which is a technology capable of delivering an active lipo-dissolving chemical compound, such as deoxycholic acid or its salts or derivatives or other chemical with detergent effects into the subcutaneous skin layer directly into the fat deposits or their proximity.

In the present invention, the deoxycholic acid or its salts or derivatives or other chemical with detergent effects which are encapsulated within or incorporated into liposomes may be associated with phoshatidylcholine and/or L-carnitine which seems to help the metabolization of fat.

### OBJECT OF THE PRESENT INVENTION

It is an object of the present invention to provide a simple, rapidly transdermally deployable topical preparation for the effective treatment of unwanted fat and lipodystrophies.

It is an object of the present invention to provide the consumer with a simple non invasive effective, rapidly deployable means and method for improving cosmetic appearance via the elimination of unwanted fat.

It is an object of the present invention to provide the consumer with a safe, simple and effective apparatus and method to target and to induce lysis on adipose cells in body areas of specific user's concern, exactly where those adipose cells aggregates are unwanted.

It is an object of the present invention to provide the consumer with a safe, simple effective topical preparation such as cream, lotion, emulsion, paste, ointment and the likes with lipo-dissolving capabilities never available before.

It is an object of the present invention to utilize a proven safe effective apparatus and method of transdermal drug delivery such as the liposome technology for targeting specifically adipose tissue accessible otherwise only via injections.

### DRAWING FIGURES

FIG. 1 illustrates a liposome in cross section encapsulating molecules of sodium deoxycholate.

FIG. 2 illustrates another embodiment of a liposome in cross section with the molecules of sodium deoxycholate bonded directly to the phosholipids wall of the liposome vesicle.

### DETAILED DESCRIPTION OF THE INVENTION

The invention consist of a topical dermatological liposome base preparation such as a cream, lotion, ointment, paste and the likes containing deoxycholic acid or its salts or its derivatives.

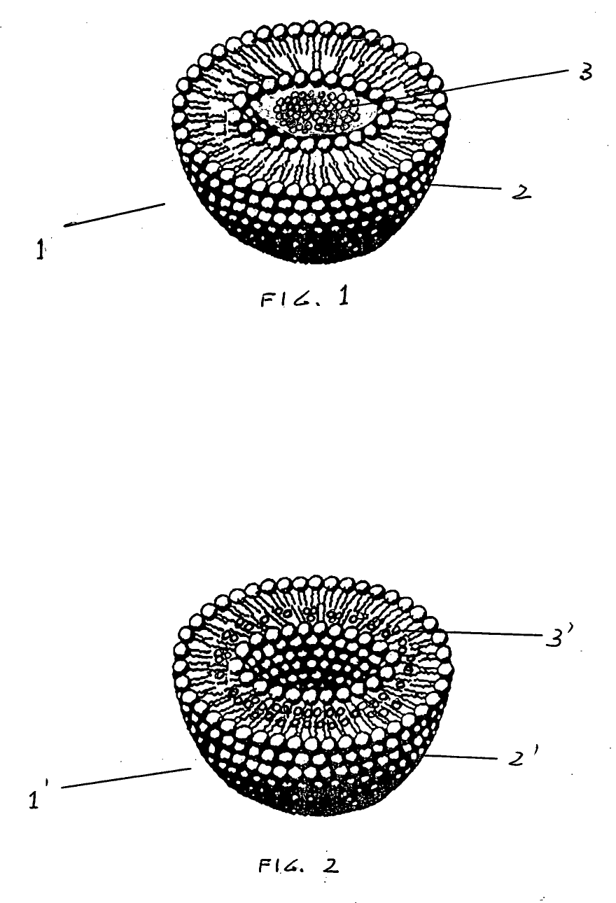

As shown in FIG. 1 liposomes, generally indicated as 1, are microscopic spherical vesicles that form when phospholipids are being hydrated.

Liposomes are typical dermatological vehicles which are able to transport dermatological active agents of different types through the skin layers. The active ingredients contained within liposome's are encapsulated and protected by the liposome's bi- or multi-layers phospholipids walls 2 as shown in FIG. 1. Liposomes, due to their physical-chemical characteristics, transverse excellently the horny impermeable layer of the skin known as stratum corneum and the whole epidermis in general. When a cosmoceutical or drug-containing liposome is applied to the skin, the liposome passes thru the outer skin layer carrying the encapsulated pay-load and releases its pay load of active material into the sub-stratum-corneum structures of the epidermis, into the dermis and into the subcutaneous tissue.

Besides being encapsulated within the liposome vesicles as shown in FIG. 1, active ingredients 3′ can be incorporated with the phospholipids wall 2′ itself of liposome 1′ as shown in FIG. 2 with a special innovative manufacturing method. As in FIG. 2, the active ingredient lipo-dissolving molecule 3′ to be carried is directly bonded to the phospholipids of the liposome lipid bi-layers or multilayers membrane or wall 2′, becoming part of it.

Liposomes, which are generally made of lecithin organogels, can incorporate sizeable amounts of quite different chemicals as guest molecules and therefore fulfill the conditions necessary for cosmetic and pharmacological applications.

Liposomes have shown that:

A) they can transport soluble substances in both water and oils.

B) they have high affinity with biological membranes and are natural and biodegradable.

C) they can cross both the dermal barrier and the cellular barrier.

Deoxycholic acid or sodium deoxycholate, a bile salt, has shown to be able of causing loss of cell viability, cell membrane lysis and disruption of fat architecture in cell cultures and tissue specimen. These findings are much similar to the effects produced after treatment with laboratory detergents.

With the present invention deoxycholic acid or sodium deoxycholate or deoxycholic acid salt or deoxycholic acid derivative or detergent substance 3 is housed, encapsulated within the liposome vesicles 1 as shown in FIG. 1, or bond to or incorporated 3′ into the liposome membrane or wall 2′ as shown in FIG. 2.

Once the liposome releases its deoxycholate payloads 3 or and 3′ into the tissue thru penetration of the horny skin layer and the epidermis, lysis of fat cells will take place in a fashion similar to lysis of fat cells occurring as a result of the action of the deoxycholate introduced percutaneously via injections. Transdermal delivery of deoxycholic acid can also be improved by the use of skin permeability enhancers such as hydrating compounds, methanol, azones, menthol, terpenes, alcohol, methanol and others. Other skin permeability enhancers are electrical means, such as electrical means inducing electroporation, and ultrasound means which also have been proved to enhance skin permeability.

Deoxycholate can also be transdermally delivered via transdermal delivery patches, some of which may actually enhance transdermal delivery by the use of electrical fields that allow water soluble compounds such as deoxycholate to migrate through the skin.

Liposomes beside carrying deoxycholic acid or sodium deoxycholate or deoxycholic acid salt or deoxycholic acid derivative or detergent substances can carry other lipo-dissolving or cell wall disrupting substances such as collagenase, lipase, bromeline or papaine.

What we claim is:

1. A topical dermatological preparation for body fat treatment comprising:
liposomes and
a substance containing deoxycholic acid, wherein said liposomes vehiculate said substance into body tissues.

2. The topical dermatological preparation of claim 1 being a cream.

3. The topical dermatological preparation of claim 1 being an ointment.

4. The topical dermatological preparation of claim 1 being a lotion.

5. The topical dermatological preparation of claim 1 being an emulsion.

6. The topical dermatological preparation of claim 1 being a paste.

7. The topical dermatological preparation of claim 1 wherein the substance is a deoxycholic acid salt.

8. The topical dermatological preparation of claim 1 wherein the substance is a chemical derivative of the deoxycholic acid.

9. The topical dermatological preparation of claim 1 wherein the substance is a detergent substance.

10. The topical dermatological preparation of claim 1 wherein the substance is encapsulated by the liposome.

11. The topical dermatological preparation of claim 1 wherein the substance is incorporated into the liposome.

12. A topical dermatological preparation comprising:
liposomes and
deoxycholic acid, wherein said liposomes vehiculate said deoxycholic acid into body tissues.

13. The substance of claim 1 being delivered by the liposomes into the adipose tissue or its proximity.
